(19) United States
(12) Patent Application Publication
(10) Pub. No.: US 2023/0154584 A1
(43) Pub. Date: May 18, 2023

(54) COMPUTER SYSTEM AND INTERVENTION EFFECT PREDICTING METHOD

(71) Applicant: Hitachi, Ltd., Tokyo (JP)

(72) Inventors: Peifei ZHU, Tokyo (JP); Masahiro OGINO, Tokyo (JP); Zisheng LI, Tokyo (JP)

(73) Assignee: Hitachi, Ltd., Tokyo (JP)

(21) Appl. No.: 17/980,709

(22) Filed: Nov. 4, 2022

(30) Foreign Application Priority Data
Nov. 12, 2021 (JP) 2021-185031

(51) Int. Cl. G16H 20/00

(52) U.S. Cl. CPC G16H 20/00

(57) ABSTRACT

A computer system manages a first model calculating an output value, using time-series data including a value related to an intervention carried out on a person, a second model that calculates a feature by mapping an output value from the first model onto a feature space, and a third model that outputs a predicted value of an effect of an intervention, from the feature. The time-series data includes a time at which the intervention is carried out, factors indicating a state of the person, and a type and a degree of the intervention. The computer system calculates a predicted value of the continuous intervention corresponding to the time-series data, using the first, second, and third models. The second model maps an output value from the first model onto the feature space so that a difference in distribution of data strings used in machine learning reduces in the feature space.

### CLAIM OF PRIORITY

The present application claims priority from Japanese patent application JP 2021-185031 filed on Nov. 12, 2021, the content of which is hereby incorporated by reference into this application.

### BACKGROUND OF THE INVENTION

### 1. Field of the Invention

The present invention relates to a system and a method that predict an effect of intervention on a person.

### 2. Description of the Related Art

In various fields, such as medical care and marketing, predicting an effect of intervention on a person (a medication effect, an exercise effect, and the like) is now required. Confounding factors are important in predicting an intervention effect. Confounding factors affect an intervention effect and are relevant to causes/factors. When observed data indicates a correlation, it is necessary to determine whether the correlation originates from a causal relationship or the influence of confounding factors.

A randomized comparative test is known as a method of adjusting confounding factors. This method requires random selection of subjects, thus posing a problem that the subjects' burden and test costs are greater. This leads to a demand for development of a technique by which causal inference is made using existing data. Concerning this technique, a technique described in JP 2019-192065 A is known.

JP 2019-192065 A includes an explanatory statement: “In order to properly verify the effect of the care intervention, similarity-based clustering of a plurality of subjects is carried out, based on their attributes, and according to clustering results, the subjects are further divided into an intervention group and a control group, and then the intervention effect is evaluated through comparison of the intervention group and the control group”.

In recent years, a technique for predicting an intervention effect in a case of continuously carrying out multiple types of interventions on a subject has been in demand. The technique described in JP 2019-192065 A, however, is incapable of processing time-series data. As a system that makes prediction using time-series data, a technique described in JP 2020-35365 A is known.

JP 2020-35365 A includes an explanatory statement: “In order to bring the subject's health condition closer to an ideal health condition, the system learns measurement values and target values of health conditions in several days in the past, and then outputs target values of health conditions, the targets values being recommended, and target achievement expectation values to present the target values and target achievement expectation values to the user.”

### SUMMARY OF THE INVENTION

The technique described in JP 2020-35365 A, however, does not take into consideration the influence of confounding factors (attributes, such as gender and age, and past intervention results).

The present invention provides a system and a method that predict an intervention effect in a case where a plurality of types of interventions are continuously carried out on a subject as the influence of confounding factors is taken into consideration.

A typical example of the present invention disclosed herein is as follows. The present invention provides a computer system that predicts an effect of a plurality of interventions on a person, the computer system including at least one computer including a processor and a storage device connected to the processor. The computer system manages a first model that calculates an output value, using time-series data including a value related to an intervention carried out on a person, a second model generated by machine learning, the second model calculating a feature by mapping an output value from the first model onto a feature space, and a third model that outputs a predicted value of an effect of an intervention on the person, based on the feature. The time-series data includes a plurality of data strings including a time at which the intervention is carried out on the person, a plurality of factors indicating a state of the person, and a type and a degree of the intervention carried out on the person. The processor executes a prediction process including: calculating the output value by inputting the data string to the first model; calculating the feature by inputting the output value to the second model; and calculating a predicted value of an effect of the intervention carried out continuously, the intervention corresponding to the time-series data, by inputting the feature to the third model. The second model maps an output value from the first model onto the feature space so that a difference in distribution of a plurality of data strings used in the machine learning reduces in the feature space.

The present invention allows predicting an intervention effect in a case where a plurality of types of interventions are continuously carried out on a subject as the influence of confounding factors are taken into consideration. Problems, configurations, and effects that are not described above will be clarified by the following description of embodiments.

### BRIEF DESCRIPTION OF THE DRAWINGS

FIG. 1 depicts a configuration example of a system of a first embodiment;

FIG. 2 depicts an example of a software configuration of a computer of the first embodiment;

FIG. 3 depicts an example of a learning data DB of the first embodiment;

FIG. 4 depicts an example of a functional configuration of a predicting unit of the first embodiment;

FIG. 5 depicts an example of a functional configuration of a learning unit of the first embodiment;

FIG. 6 is a flowchart for explaining an example of a learning process executed by the learning unit of the first embodiment;

FIG. 7 is a flowchart for explaining an example of a prediction process executed by the predicting unit of the first embodiment;

FIG. 8 depicts an example of a screen presented by the predicting unit of the first embodiment;

FIG. 9 is a flowchart for explaining an example of a prediction process executed by the predicting unit of a second embodiment; and

FIG. 10 depicts an example of a screen presented by the predicting unit of the second embodiment.

### DESCRIPTION OF THE PREFERRED EMBODIMENTS

Embodiments of the present invention will hereinafter be described with reference to the drawings. It should be noted, however, that the present invention is not interpreted as the invention limited to the descriptive contents of embodiments described below. Those skilled in the art can easily understand that specific configurations of the invention may be changed or modified within a range in which changes/modification do not deviate from the concept and substance of the present invention.

In the configurations of the invention described below, the same or similar constituent elements or functions are denoted by the same reference signs, and redundant description will be omitted.

In this specification, such notations as “first”, “second”, and “third” are attached to constituent elements to identify them, and do not necessarily limit the number or order thereof.

The positions, sizes, shapes, ranges, and the like of constituent elements shown in drawings, etc., may not represent the actual positions, sizes, shapes, ranges, and the like. This is to facilitate understanding of the invention. The constituent elements of the present invention, therefore, are not limited by the positions, sizes, shapes, ranges, and the like shown in the drawings, etc.

### First Embodiment

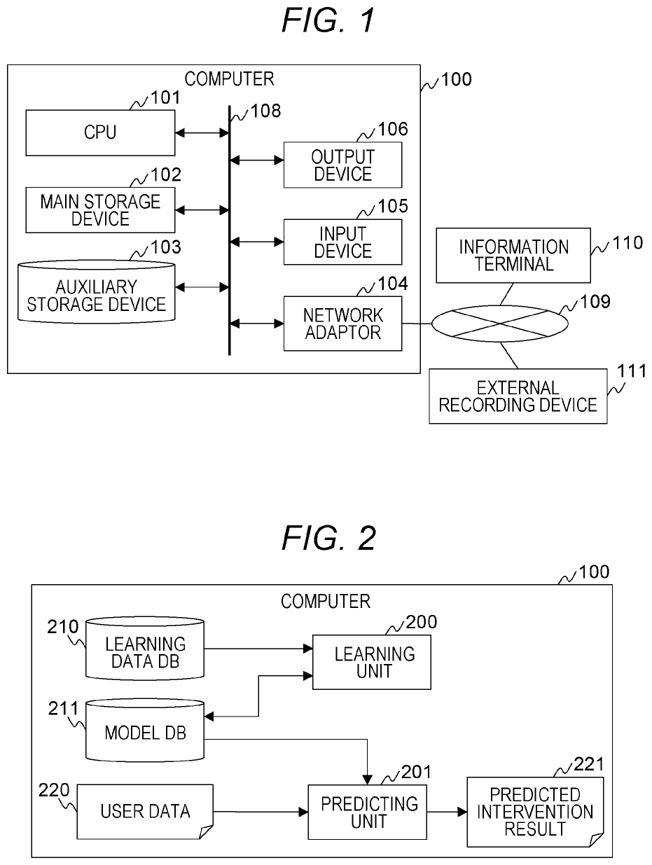

FIG. 1 depicts a configuration example of a system of a first embodiment.

The system includes a computer 100, an information terminal 110, and an external storage device 111. The computer 100, the information terminal 110, and the external storage device 111 are interconnected via a network 109. The network 109 is, for example, a local area network (LAN), a wide area network (WAN), or the like, and provides wired connection or wireless connection.

The computer 100 executes a learning process for generating a model for predicting an intervention effect, and predicts an intervention effect on user data (input data), using the model. The computer 100 includes a CPU 101, a main storage device 102, an auxiliary storage device 103, a network adapter 104, an input device 105, and an output device 106. These hardware elements are connected to each other via an internal bus 108.

The CPU 101 runs a program stored in the main storage device 102. The CPU 101 executes a process according to a program, thus working as a functional unit (module) that exerts a specific function. In the following description, when a process is described with a functional unit used as the subject of the description, it means that the CPU 101 is executing a program for providing the functional unit.

The main storage device 102 is a dynamic random access memory (DRAM), storing programs executed by the CPU 101 and data used by the programs. In addition, the main storage device 102 is used as a work area.

The auxiliary storage device 103 is a hard disk drive (HDD), a solid state drive (SSD), or the like, storing data permanently. Programs and data to be stored in the main storage device 102 may be stored in the auxiliary storage device 103. In such a case, the CPU 101 reads a program and information from the auxiliary storage device 103 and loads the program and information onto the main storage device 102.

The network adapter 104 is an interface for connecting to an external device via the network 109.

The input device 105 is a keyboard, a mouse, a touch panel, or the like, serving as a device for making data input to the computer 100.

The output device 106 is a display, a printer, or the like, serving as a device for outputting process results etc., from the computer 100.

It should be noted that the above hardware configuration of the computer 100 is an exemplary one and therefore the hardware configuration of the computer 100 is not limited to the above configuration. For example, the computer 100 may include neither the input device 105 nor the output device 106.

The information terminal 110 carries out various operations on the computer 100. For example, the information terminal 110 carries out registration of learning data, registration of a model, input of user data, and the like. The information terminal 110 is the same in hardware configuration as the computer 100.

The external storage device 111 stores various pieces of information. The external storage device 111 is, for example, an external HDD or a storage system.

FIG. 2 depicts an example of a software configuration of the computer 100 of the first embodiment.

The computer 100 includes a learning unit 200 and a predicting unit 201, and further includes a learning data DB 210 and a model DB 211. The learning data DB 210 and the model DB 211 may be stored in the external storage device 111.

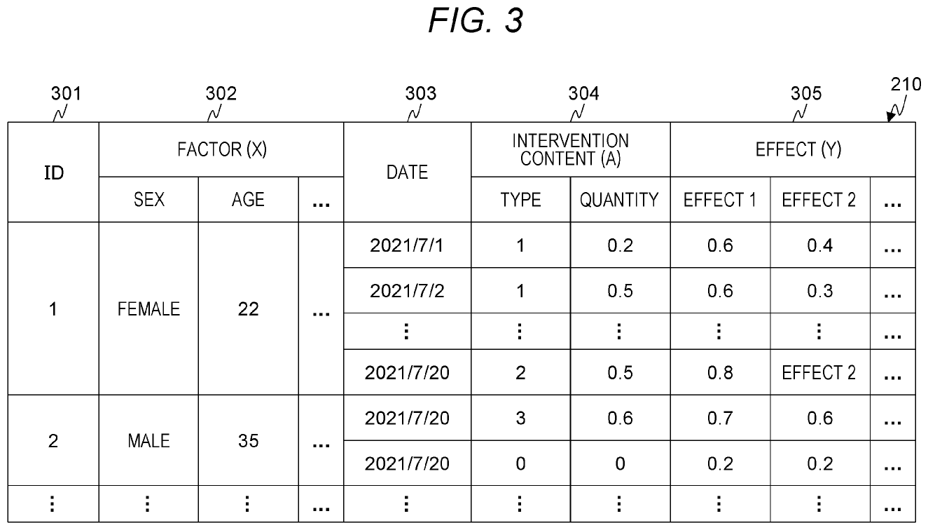

The learning data DB 210 stores learning data used for a learning process. The learning data DB 210 will be described with reference to FIG. 3. The model DB 211 stores information on various models.

The learning unit 200 executes a learning process, using learning data stored in the learning data DB 210 and a model stored in the model DB 211. The predicting unit 201 predicts an intervention effect on user data 220, using a model stored in the model DB 211, and outputs the predicted intervention effect as a predicted intervention result 221. It should be noted that the learning data and the user data 220 of this embodiment are time-series data.

FIG. 3 depicts an example of the learning data DB 210 of the first embodiment.

The learning data DB 210 stores entries of ID 301, factor 302, date 303, intervention content 304, and effect 305. One entry corresponds to one piece of learning data. Fields included in entries are not limited to the fields described above as the entries. The learning data DB 210 may not include one of the above fields or may include other fields different from the above fields.

The ID 301 is a field storing identification information for uniquely identifying learning data. Identification numbers are stored in the ID 301 of this embodiment.

The factor 302 is a field storing values of factors, such as the condition and characteristics of a person who undergoes intervention. Factors include, for example, age, sex, and height. In this embodiment, the types and number of factors are not limited to the types and number of factors included in the factor 302.

Learning data of this embodiment is time-series data. One piece of learning data, therefore, includes a plurality of data strings composed of data of the date 303, the intervention content 304, and the effect 305.

The date 303 is a field storing dates. The date 303 stores the date of measurement of an intervention effect or the date of generation of a data string. According to the present invention, date entries are not limited to the type of date entries made in the date 303. Any type of date entries that allows understanding of a time-series flow is applicable to the present invention.

The intervention content 304 is a field group storing information indicating the content of intervention carried out on a person. The intervention content 304 includes fields of type and quantity. The type is a field storing types of interventions. The type stores, for example, values representing types of medicine, treatment, exercise, etc. The quantity is a field storing values representing degrees of intervention. For example, a value representing a dose of medicine, exercise time, or the like is stored in the quantity. In this embodiment, when no intervention is carried out, 0 is entered in the type and in the quantity.

The effect 305 is a field group storing values of indexes indicating intervention effects (effect predicted values). In this embodiment, the type and number of indexes are not limited to the type and number of indexes included in the effect 305.

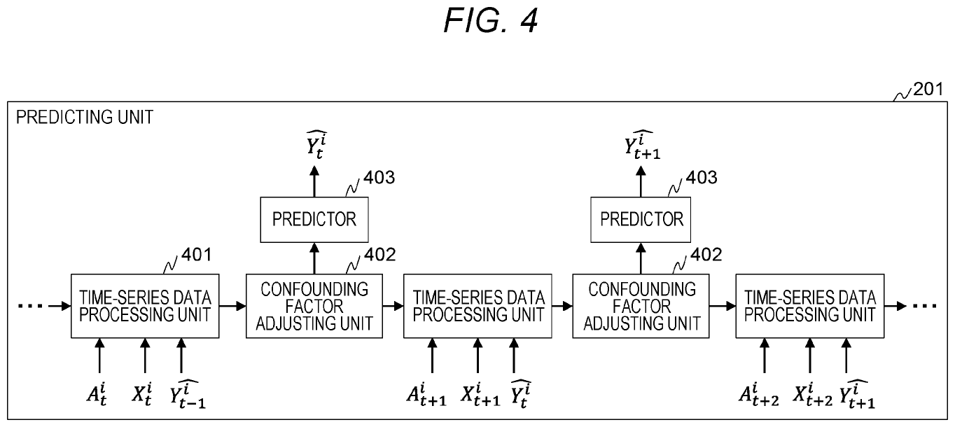

FIG. 4 depicts an example of a functional configuration of the predicting unit 201 of the first embodiment.

The predicting unit 201 includes a time-series data processing unit 401, a confounding factor adjusting unit 402, and a predictor 403.

The time-series data processing unit 401 calculates an output value, using time-series data. The time-series data processing unit 401 is, for example, a recurrent neural network (RNN). The RNN is a type of neural network, and is characterized in that it generates input and output at each time step. Time step intervals can be set arbitrarily. The RNN uses output from the previous time step as new input, thereby obtaining output for which a time series flow is taken into consideration. For output from the RNN, however, the influence of confounding factors is not taken into consideration.

At time step t, the time-series data processing unit 401 of this embodiment receives intervention content and a factor at time step t and an effect predicted value at time step (t−1), as inputs. In a case of t=0, the time-series data processing unit 401 receives inputs of only the intervention content and factor at a point of t=0.

In the present specification, for a person with identification information i, intervention content at time step t is defined as Aₜⁱ, a factor at time step t is defined as Xₜⁱ and an effect predicted value at time step t is defined as Y\hat{ }ₜⁱ. A person with identification information i is expressed as a person (i). It should be noted that A\hat{ } and Y\hat{ }correspond to A and Y with a hat symbol appended thereto in mathematical formulas and drawings.

At time step t, the time-series data processing unit 401 calculates an output value (feature), using the intervention content Aₜⁱ and factor Xₜⁱ at time step t and the effect predicted value Y\hat{ }₁₋₁ⁱ at time step (t−1). According to this embodiment, the output value is a vector value.

To achieve more accurate prediction of an intervention effect, the confounding factor adjusting unit 402 executes a process of reducing the influence of confounding factors on the output value.

Confounding factors of this embodiment are classified into two categories: factors and effects of interventions carried out in the past. Regarding the influence of factors, for example, a case where young people often select an intervention 1 while elderly people often select an intervention 2 is considered. In this case, an accurate effect cannot be predicted because of an age distribution bias included in the intervention 1 and the intervention 2. In this case, it is hard to tell whether the effect is given by the interventions or results from biased factors. Regarding the influence of the effect of interventions carried out in the past, for example, a case where a person who was given medicine 1 in the previous intervention suffers from an aftereffect is considered. In this case, a possibility of choosing the medicine 1 is low. This case leads to a biased distribution of people who select a specific medicine, and therefore affects the accuracy of effect prediction.

The confounding factor adjusting unit 402 carries out a process of reducing distribution differences in such a way as to give every intervention an equal chance to be selected, thus generates a feature with a balanced distribution. Specifically, the confounding factor adjusting unit 402 maps an output value (vector value) calculated by the time-series data processing unit 401, onto a feature space of any given dimension, thereby determining the feature.

The predictor 403 calculates an effect predicted value of an intervention, using a feature calculated by the confounding factor adjusting unit 402. The predictor 403 is, for example, a neural network or a linear regression model.

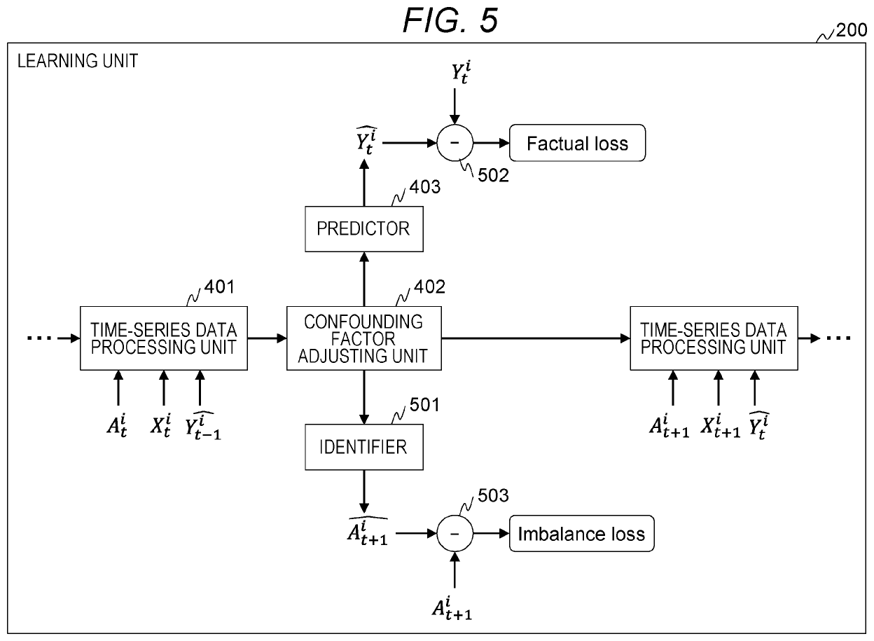

FIG. 5 depicts an example of a functional configuration of the learning unit 200 of the first embodiment.

The learning unit 200 includes the time-series data processing unit 401, the confounding factor adjusting unit 402, the predictor 403, an identifier 501, an arithmetic unit 502, and an arithmetic unit 503. The time-series data processing unit 401, the confounding factor adjusting unit 402, and the predictor 403 are the same as those included in the predicting unit 201. The learning unit 200 trains the confounding factor adjusting unit 402, the predictor 403, and the identifier 501 by using such a learning method as Adversarial Learning.

The identifier 501 receives input of a feature calculated by the confounding factor adjusting unit 402, and predicts intervention content A\hat{ }₁₊ⁱ that is the content of an intervention to be carried out on a person (i) at the next time step (t+1). The identifier 501 is defined as a neural network model, etc.

The arithmetic unit 503 calculates an imbalance loss for evaluating an error between the predicted intervention content A\hat{ }ₜ₊₁ⁱ and actual intervention content Aₜ₊₁ⁱ. An imbalance loss function for calculating the imbalance loss is defined by equation (1).

### [Equation 1]

In equation 1, G_{g} denotes a function representing output from the confounding factor adjusting unit 402, and G_{d} denotes a function representing output from the identifier 501. n denotes the number of fields (the number of samples) the factor 302 has. II denotes an indication function, κ denotes a threshold, denotes an error tolerance, and N denotes the number of samples within a range (Aₜ₊₁^{j+}ε) with κ at its center.

To allow calculation on multiple types of interventions, the continuity of calculation needs to be ensured. In a case where a difference between intervention content Aₜ₊₁ⁱ and intervention content Aₜ₊₁ⁱ is equal to or smaller than the threshold κ and a case where the difference is equal to or larger than the threshold κ, therefore, an intervention content prediction error is multiplied by different weights in both cases, respectively, to calculate the Imbalance loss. The intervention content prediction error corresponds to a logarithmic term of equation (1).

The learning unit 200 trains the identifier 501 so as to increase prediction accuracy, and trains the confounding factor adjusting unit 402 as well so that the identifier 501 cannot discriminate against any factor.

The arithmetic unit 502 calculates a factual loss for evaluating an error between an effect predicted value Y\hat{ }ₜ₋₁ⁱ calculated by the predictor 403 and an actual intervention effect Yₜ₋₁ⁱ. A factual loss function for calculating the factual loss is defined by equation (2).

### [Equation 2]

In equation 2, G_{y} denotes a function representing output from the predictor 403.

As shown in equation (3), the learning unit 200 trains each model so that a loss function takes a minimum value, the loss function being defined by the sum of imbalance losses at all time steps and the sum of factual losses at all time steps. In the learning process, the learning unit 200 updates the identifier 501 so that the accuracy of intervention content prediction based on the feature is improved, and updates the confounding factor adjusting unit 402 so that the intervention content of the identifier 501 cannot be predicted.

### [Equation 3]

In equation 3, α denotes a parameter for adjusting the factual loss and the imbalance loss.

By the learning process using the loss function, a difference in distribution of features generated by the confounding factor adjusting unit 402 can be reduced. In other words, the influence of confounding factors can be reduced. As a result, an intervention effect can be predicted accurately.

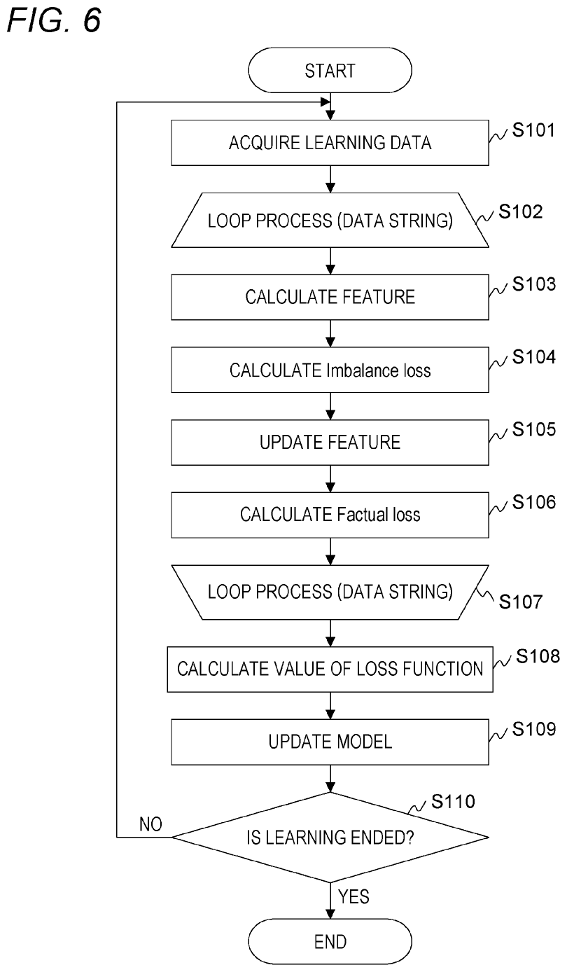

FIG. 6 is a flowchart for explaining an example of a learning process executed by the learning unit 200 of the first embodiment.

When receiving a learning execution instruction via the information terminal 110 or the input device 105, the learning unit 200 executes the learning process.

The learning unit 200 acquires learning data from the learning data DB 210 (step S101). It is assumed in this case that a learning data set composed of pieces of learning data is acquired.

Subsequently, the learning unit 200 starts a loop process on data strings included in the learning data (step S102). The learning unit 200 selects data strings in time-series order, and repeatedly executes the following steps.

The learning unit 200 calculates a feature, using a data string (step S103). Specifically, the learning unit 200 inputs the intervention content and factor that correspond to the data string and an effect predicted value obtained by using a data string one time step before in the time-series order, to the time-series data processing unit 401, and inputs an output value calculated by the time-series data processing unit 401, to the confounding factor adjusting unit 402. The learning unit 200 stores the feature associated with the time-series order, in the work area.

The learning unit 200 inputs the feature to the identifier 501, and calculates an imbalance loss, based on predicted intervention content A\hat{ }ₜ₊₁ⁱ outputted from the identifier 501 and on intervention content Aₜ₊₁ⁱ of a data string one time step ahead in the time-series order (step S104). The learning unit 200 stores the imbalance loss associated with the time-series order, in the work area.

The learning unit 200 updates the identifier 501 and the confounding factor adjusting unit 402 by a back error propagation method using the imbalance loss function or the like, and updates the feature through the updated confounding factor adjusting unit 402 (step S105).

The learning unit 200 inputs the updated feature to the predictor 403, and calculates a factual loss, based on intervention effect predicted value Y\hat{ }ₜⁱ outputted from the predictor 403 and on intervention effect Yₜⁱ on the data string (step S106). The learning unit 200 stores the factual loss associated with the time-series order, in the work area.

The learning unit 200 determines whether it has completed the steps on all data strings included in the learning data (step S107).

When not completing the steps on all data strings included in the learning data, the learning unit 200 returns to step S102, and executes the same steps again.

When completing the steps on all data strings included in the learning data, the learning unit 200 calculates a value of the loss function expressed by equation (3) (step S108).

Based on the value of the loss function, the learning unit 200 updates the confounding factor adjusting unit 402, the predictor 403, and the identifier 501 (step S109).

The learning unit 200 determines whether or not to end the learning process (step S110). For example, when completing the processes on all data strings included in the learning data, the learning unit 200 determines to end the learning process. When the number of times of updating is larger than a threshold, the learning unit 200 determines to end the learning process. When the accuracy of prediction of an intervention effect on the user data 220 for evaluation is higher than a threshold, the learning unit 200 determines to end the learning process.

When determining not to end the learning process, the learning unit 200 returns to step S101 and executes the same steps again.

When determining to end the learning process, the learning unit 200 ends the learning process.

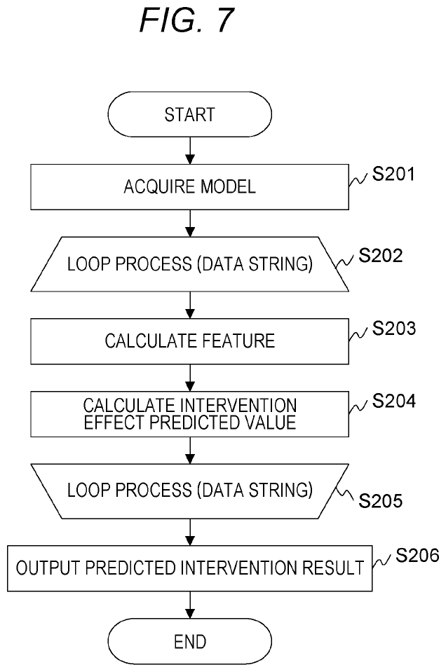

FIG. 7 is a flowchart for explaining an example of a prediction process executed by the predicting unit 201 of the first embodiment.

When receiving a prediction execution instruction including the user data 220 via the information terminal 110 or the input device 105, the predicting unit 201 executes prediction process.

The predicting unit 201 acquires models of the time-series data processing unit 401, the confounding factor adjusting unit 402, and the predictor 403, from the model DB 211 (step S201).

The predicting unit 201 starts a loop process on data strings included in the user data 220 (step S202). The predicting unit 201 selects data strings in time-series order, and repeatedly executes the following steps.

The predicting unit 201 calculates a feature, using a data string (step S203). Specifically, the predicting unit 201 inputs the intervention content and factor that correspond to the data string and an effect predicted value obtained by using a data string one time step before in the time-series order, to the time-series data processing unit 401, and inputs an output value calculated by the time-series data processing unit 401, to the confounding factor adjusting unit 402. The predicting unit 201 stores the feature associated with the time-series order, in the work area.

The predicting unit 201 inputs the feature to the predictor 403, thereby calculating an intervention effect predicted value (step S204).

The predicting unit 201 determines whether it has completed the steps on all data strings included in the user data 220 (step S205).

When not completing the steps on all data strings included in the user data 220, the predicting unit 201 returns to step S202, and executes the same steps again.

When completing the steps on all data strings included in the user data 220, the predicting unit 201 generates and outputs a predicted intervention result 221 including intervention effect predicted values corresponding respectively to data strings (step S206). The predicting unit 201 then ends the prediction process.

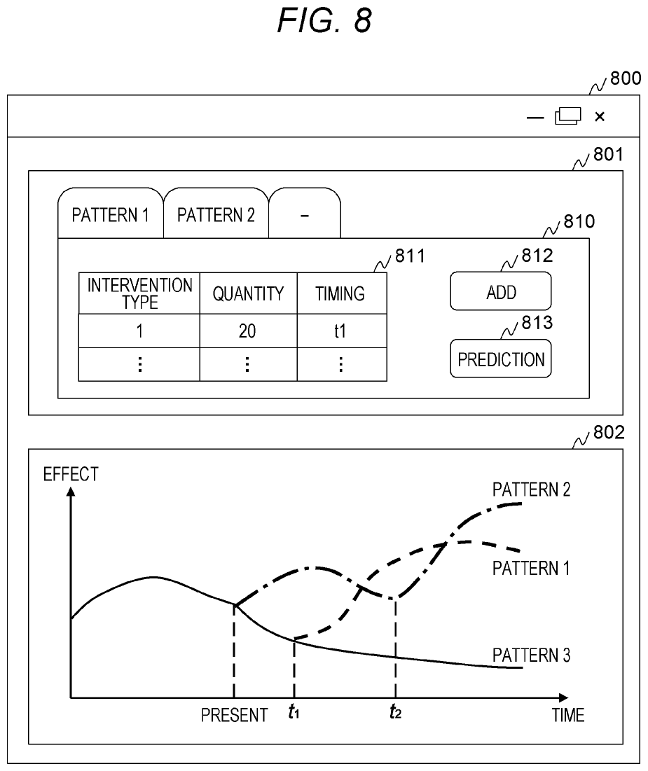

A screen presented by the predicting unit 201 will then be described. FIG. 8 depicts an example of a screen presented by the predicting unit 201 of the first embodiment.

The predicting unit 201 presents a screen 800 to the user. The screen 800 includes an intervention content input space 801 and an intervention effect display space 802.

In the intervention content input space 801, a pattern setting space 810 for inputting an intervention pattern is displayed in a tab format. The pattern setting space 810 includes a setting table 811, an addition button 812, and a prediction button 813. The setting table 811 is a table for setting intervention content, and stores entries of intervention type, quantity, and timing. The addition button 812 is an operation button for adding an entry to the setting table 811. The prediction button 813 is an operation button for giving an instruction on execution of a prediction process. When the prediction button 813 is pressed, the user data 220, which includes time-series data up to the present point and information provided by the setting table 811, is inputted to the predicting unit 201.

The intervention content may be set in a setting form different from the setting form of the pattern setting space 810 of FIG. 8. For example, a setting form may be adopted in which the intervention type is displayed on a pull-down menu as the quantity and the timing are displayed and controlled on a control bar.

The intervention effect display space 802 is a space in which transitions in effects and intervention effects from the past to the present are displayed. In the intervention effect display space 802, graphs showing transitions in intervention effects are displayed respectively for intervention patterns. FIG. 8 shows transitions in an intervention effect achieved by an intervention pattern 1 in which an intervention 1 is carried out at time t₁, transitions in an intervention effect achieved by an intervention pattern 2 in which an intervention 2 is carried out at the present point and time t2, and transitions in an intervention effect achieved by an intervention pattern 3 in which no intervention is carried out.

The system of the first embodiment can reduce the influence of confounding factors and highly accurately predict an effect achieved by continuously carrying out a plurality of types of interventions on a person.

When the input content of the intervention content input space 801 is updated, the display content of the intervention effect display space 802 is too updated. The intervention effect display space 802 may display only the transitions in an effect achieved by a specific intervention pattern.

### Second Embodiment

A system according to a second embodiment carries out a prediction process again when an intervention effect predicted value is corrected. The second embodiment will hereinafter be described, with focus placed on a difference with the first embodiment.

The system of the second embodiment is the same in configuration as the system of the first embodiment. The functional configurations of the learning unit 200 and the predicting unit 201 of the second embodiment are the same as those of the first embodiment. The process executed by the learning unit 200 of the second embodiment is the same as the process executed by the learning unit 200 of the first embodiment.

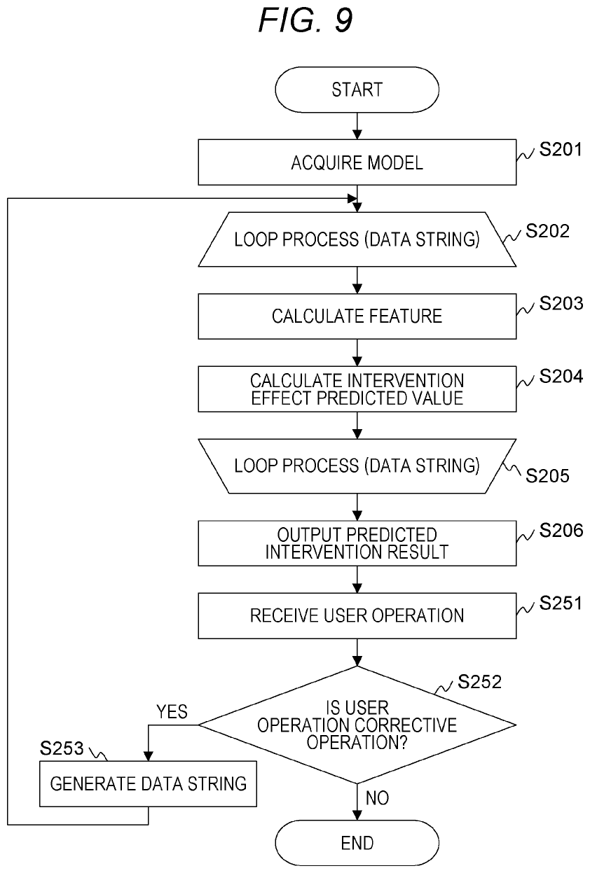

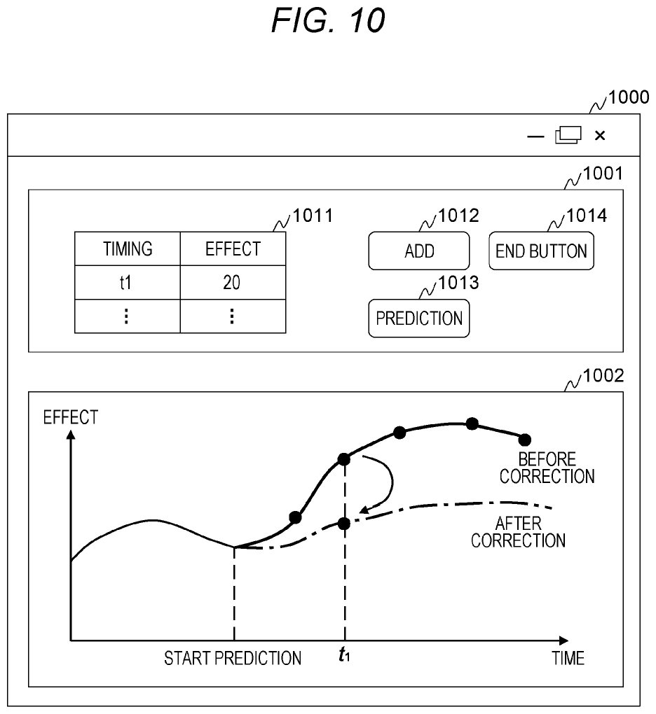

In the second embodiment, a prediction process executed by the predicting unit 201 is partially different from the prediction process in the first embodiment. FIG. 9 is a flowchart for explaining an example of the prediction process executed by the predicting unit 201 of the second embodiment. FIG. 10 depicts an example of a screen presented by the predicting unit 201 of the second embodiment.

Step S201 to step S206 are the same as step S201 to step S206 in the first embodiment. Following execution of step S206, the predicting unit 201 presents the screen on which the user's operation is received (step S251). The screen presented by the predicting unit 201 will then be described with reference to FIG. 10.

The predicting unit 201 presents a screen 1000 to the user. The screen 1000 includes a correction space 1001 and an intervention effect display space 1002. The intervention effect display space 1002 is the same as the intervention effect display space 802.

The correction space 1001 includes a correction setting table 1011, an addition button 1012, a prediction button 1013, and an end button 1014. The correction setting table 1011 is a table for setting corrective content of an intervention effect predicted value, and stores entries of timing and effect. The addition button 1012 is an operation button for adding an entry to the correction setting table 1011. The prediction button 1013 is an operation button for giving an instruction on re-execution of the prediction process. When the prediction button 1013 is pressed, the corrective content is inputted to the predicting unit 201. The end button 1014 is an operation button for ending the prediction process.

The corrective content may be set in a setting form different from the setting form of the correction space 1001 of FIG. 10. For example, a correction button is provided, which is pressed to display a corrective point on a graph displayed in the intervention effect display space 1002. The user corrects the intervention effect predicted value by manipulating the point displayed on the graph, using a mouse or the like.

The description of the screen is ended here. Now the flowchart of FIG. 9 will be described again.

The predicting unit 201 determines whether an operation received on the screen 1000 is a correction operation.

When the received operation is an end operation, the predicting unit 201 ends the prediction process.

When the received operation is a correction operation, the predicting unit 201 generates a data string to be used for a new prediction process (step S253), and then returns to step S202. For example, the predicting unit 201 generates a data string reflecting the corrected intervention effect predicted value.

It should be noted that the present invention is not limited to the above embodiments but includes various modifications. For example, the above embodiments have been described in detail for easy understanding of the present invention, and are not necessarily limited to an embodiment including all the constituent elements described above. In addition, some of constituent elements of each embodiment can be deleted therefrom or add to or replaced with constituent elements of another embodiment.

Some or all of the above constituent elements, functions, processing units, processing means, and the like may be provided as hardware, such as properly designed integrated circuits. The present invention may be embodied by a software program code that implements the functions of the embodiments. In such a case, a computer is provided with a storage medium recording the program code, and a processor incorporated in the computer reads the program code from the storage medium. In this case, the program code itself, which is read from the storage medium, implements the above functions of the embodiments, and the program code and the storage medium storing the program code constitute the present invention. Storage media for supplying such a program code include, for example, a flexible disk, a CD-ROM, a DVD-ROM, a hard disk, a solid state drive (SSD), an optical disk, a magneto-optical disk, a CD-R, a magnetic tape, a nonvolatile memory card, and a ROM.

The program code that implements the functions described in the present invention can be written in a wide variety of program or script languages, such as assembler, C/C++, perl, Shell, PHP, Python, and Java (registered trademark).

The software program code that implements the functions of the embodiments may be distributed via a network, in which case the program code is stored in a storage means, such as a hard disk or a memory, of a computer or in a storage medium, such as a CD-RW or a CD-R, and a processor incorporated in the computer reads the program code from the storage means or the storage medium to execute the program code.

In the above embodiments, a group of control lines/data lines considered to be necessary for description are illustrated, and all control lines/information lines making up the product are not always illustrated. All constituent elements may be interconnected.

1. A computer system that predicts an effect of a plurality of interventions on a person, the computer system comprising at least one computer including a processor and a storage device connected to the processor,
wherein the computer system manages a first model that calculates an output value, using time-series data including a value related to an intervention carried out on a person, a second model generated by machine learning, the second model calculating a feature by mapping an output value from the first model onto a feature space, and a third model that outputs a predicted value of an effect of an intervention on the person, based on the feature,
wherein the time-series data includes a plurality of data strings including a time at which the intervention is carried out on the person, a plurality of factors indicating a state of the person, and values indicating a type and a degree of the intervention carried out on the person,
wherein the processor executes a prediction process including:
calculating the output value by inputting the data string to the first model;
calculating the feature by inputting the output value to the second model; and
calculating a predicted value of an effect of the intervention carried out continuously, the intervention corresponding to the time-series data, by inputting the feature to the third model, and
wherein the second model maps an output value from the first model onto the feature space so that a difference in distribution of a plurality of data strings used in the machine learning reduces in the feature space.

2. The computer system according to claim 1,
wherein the computer system manages a fourth model that identifies a type of the intervention carried out on the person, from the feature, and a loss function defined by a predicted type of the intervention outputted by the fourth model, a type of the intervention included in learning data, a predicted value of an effect of the intervention, and an effect value of the intervention included in the learning data, and
wherein the processor executes the machine learning including:
receiving the learning data including a plurality of data strings including identification information on the person, a time at which the intervention is carried out on the person, values of the plurality of factors of the person, a type and a degree of the intervention the person has undergone, and an effect value of the intervention;
inputting the data string to the first model and inputting the output value outputted from the first model, to the second model;
calculating a predicted value of an effect of the intervention by inputting the feature outputted from the second model, to the third model;
calculating a predicted type of the intervention by inputting the feature outputted from the second model, to the fourth model;
calculating a value of the loss function, using a type of the intervention and an effect value of the intervention in each of the plurality of data strings, and a predicted type of the intervention and a predicted value of an effect of the intervention that are calculated from each of the plurality of data strings; and
updating the second model, the third model, and the fourth model, using the value of the loss function.

3. The computer system according to claim 2, wherein the loss function is defined by a first loss function that evaluates a sum of errors between an effect value of the intervention, the effect value being included in the data string, and a predicted value of an effect of the intervention, the predicted value being calculated from the data string, and by a second loss function that evaluates a sum of errors between a type of the intervention, the type being included in the data string, and a predicted type of the intervention, the predicted type being calculated from the data string.

4. The computer system according to claim 1,
wherein the processor presents a first user interface for adjusting a type and a degree of the intervention in at least one data string included in the time-series data and a timing of carrying out the intervention, and
wherein the processor executes the prediction process, using the time-series data including a data string inputted through the first user interface.

5. The computer system according to claim 1,
wherein the processor presents a second user interface for displaying a predicted value of an effect of the intervention, the predicted value being calculated from each of the plurality of data strings,
wherein the processor receives corrective content of the predicted value of the effect of the intervention through the second user interface, and
wherein the processor executes the prediction process, using the time-series data including a data string reflecting the corrective content of the predicted value of the effect of the intervention, the corrective content being inputted through the second user interface.

6. An intervention effect predicting method for predicting an effect of a plurality of interventions on a person executed by a computer system,
wherein the computer system includes at least one computer including a processor and a storage device connected to the processor,
wherein the computer system manages a first model that calculates an output value, using time-series data including a value related to an intervention carried out on a person, a second model generated by machine learning, the second model calculating a feature by mapping an output value from the first model onto a feature space, and a third model that outputs a predicted value of an effect of an intervention on the person, from the feature, and
wherein the time-series data includes a plurality of data strings including a time at which the intervention is carried out on the person, a plurality of factors indicating a state of the person, and a type and a degree of the intervention carried out on the person,
the intervention effect predicting method comprising causing the processor to execute a prediction process including:
calculating the output value by inputting the data string to the first model;
calculating the feature by inputting the output value to the second model; and
calculating a predicted value of an effect of the intervention carried out continuously, the intervention corresponding to the time-series data, by inputting the feature to the third model,
wherein the second model maps an output value from the first model onto the feature space so that a difference in distribution of a plurality of data strings used in the machine learning reduces in the feature space.

7. The intervention effect predicting method according to claim 6,
wherein the computer system manages a fourth model that identifies a type of the intervention carried out on the person, from the feature, and a loss function defined by a predicted type of the intervention outputted by the fourth model, a type of the intervention included in learning data, a predicted value of an effect of the intervention, and an effect value of the intervention included in the learning data,
wherein the intervention effect predicting method comprising causing the processor to execute the machine learning including:
receiving the learning data including a plurality of data strings including identification information on the person, a time at which the intervention is carried out on the person, values of the plurality of factors of the person, a type and a degree of the intervention the person has undergone, and an effect value of the intervention;
inputting the data string to the first model and inputting the output value outputted from the first model, to the second model;
calculating a predicted value of an effect of the intervention by inputting the feature outputted from the second model, to the third model;
calculating a predicted type of the intervention by inputting the feature outputted from the second model, to the fourth model;
calculating a value of the loss function, using a type of the intervention and an effect value of the intervention in each of the plurality of data strings, and a predicted type of the intervention and a predicted value of an effect of the intervention that are calculated from each of the plurality of data strings; and
updating the second model, the third model, and the fourth model, using the value of the loss function.

8. The intervention effect predicting method according to claim 7, wherein the loss function is defined by a first loss function that evaluates a sum of errors between an effect value of the intervention, the effect value being included in the data string, and a predicted value of an effect of the intervention, the predicted value being calculated from the data string, and by a second loss function that evaluates a sum of errors between a type of the intervention, the type being included in the data string, and a predicted type of the intervention, the predicted type being calculated from the data string.

9. The intervention effect predicting method according to claim 6, further comprising causing the processor to:
present a first user interface for adjusting a type and a degree of the intervention in at least one data string included in the time-series data and a timing of carrying out the intervention; and
execute the prediction process, using the time-series data including a data string inputted through the first user interface.

10. The intervention effect predicting method according to claim 6, further comprising causing the processor to:
present a second user interface for displaying a predicted value of an effect of the intervention, the predicted value being calculated from each of the plurality of data strings;
receiving corrective content of the predicted value of the effect of the intervention through the second user interface; and
execute the prediction process, using the time-series data including a data string reflecting the corrective content of the predicted value of the effect of the intervention, the corrective content being inputted through the second user interface.
